(19) United States
(12) Patent Application Publication
(10) Pub. No.: US 2021/0338317 A1
(43) Pub. Date: Nov. 4, 2021

(54) DEVICES AND METHODS FOR ABLATING BIOLOGICAL TISSUE

(71) Applicant: In Medical Group Pty Ltd, Victoria (AU)

(72) Inventors: David Morris, New South Wales (AU); Sarah Valle, New South Wales (AU)

(21) Appl. No.: 17/270,253

(22) PCT Filed: Aug. 20, 2019
(86) PCT No.: PCT/AU2019/050880

§ 371 Date: Feb. 22, 2021

(30) Foreign Application Priority Data
Aug. 21, 2018 (AU) 2018903074

(51) Int. Cl. A61B 18/14; A61B 18/12

(52) U.S. Cl. CPC A61B 18/1482; A61B 2018/00083; A61B 18/1206

(57) ABSTRACT

Disclosed herein is a tissue ablation device comprising a sheath and a probe. The sheath is positionable within body tissue and comprises a distal end, a proximal end and a lumen extending therebetween. The probe comprises an elongate portion configured to be slidably received in the lumen, the elongate portion housing an electrode that is deployable from a distal end of the probe's elongate portion into a substantially planar deployed configuration when the distal end of the elongate portion is located at or beyond the distal end of the sheath. An angle of deployment of the electrode from the distal end of the probe (and hence into the body tissue, in use) is selectable by orientating the probe with respect to the sheath.

### TECHNICAL FIELD

The present invention relates to devices, methods and systems for ablating biological tissue.

### BACKGROUND ART

Tumours (both malignant and benign) in various body organs such as the liver are often not able to be surgically removed and it is therefore necessary to treat the tumour in situ. A number of techniques are known for such in situ treatments, including devices that use radio frequency (RF) to generate heat capable of ablating biological tissue in proximity to the device.

Monopolar RF ablating devices are designed to be inserted into the target tissue (typically directly into the tumour) and ablate the tissue from the inside out upon application of an electrical field between the device and a grounding pad positioned on the patient's skin. These monopolar devices may, however, be of limited use in clinical settings because they can be overly complex and difficult to use, and require time consuming procedures that can lead to auxiliary injury to patients through grounding pad burns. Further, monopolar tissue ablation devices are often limited in the scope and size of the ablation that can be created, may exhibit poor consistency of ablative results (e.g. uneven heating of the target tissue, especially if a heat sink (e.g. a blood vessel) is close to the device) and present a risk of tumour seeding due to penetration and retraction from malignant tissue.

In light of the deficiencies of such monopolar RF ablation devices, one of the present inventors was an inventor of the multiple-electrode tissue ablation system that is described in detail in U.S. Pat. No. 9,060,782, the disclosure of which is herein incorporated in its entirety. In short, the ablation devices described in U.S. Pat. No. 9,060,782 can be positioned with the tumour therebetween such that the application of an electrical field between the devices' electrodes results in a defined energy envelope that is substantially confined to the target area (i.e. the tumour). As described in U.S. Pat. No. 9,060,782 in detail, this system can overcome numerous issues associated with conventional monopolar RF ablation because an outside to inside heating occurs, with a consequently high energy transfer to the target tissue. The high energy transfer enables ablation of tissue, even in proximity to heat sinks (e.g. blood vessels), while the defined energy envelope controls potential runaway by keeping the energy confined to the targeted area. In effect, substantially all of the applied energy goes into the target area, instead of radiating outwardly (i.e. towards a grounding plate). The combination of high energy delivery into the target area, energy delivery at the surface of the target tissue volume, as well as a high and more uniform energy density helps the devices of U.S. Pat. No. 9,060,782 to produce faster, more uniform, and more repeatable ablations.

The ablation devices described in U.S. Pat. No. 9,060,782 can be used to ablate larger tumours than is possible using other ablation techniques (e.g. monopolar, microwave, multipolar and, irreversible electroporation techniques, for example, have difficulty creating ablation zones large enough to treat tumours of 3 cm or greater), and with fewer potential complications. Indeed, this technology has proven clinically effective for ablating tumours (including hepatocellular carcinoma, colorectal cancer hepatic metastases, liver metastases, gallbladder carcinoma or hepatic adenoma) of up to about 7 cm in diameter, and is presently in clinical use throughout the world under the brand INCIRCLE.

### SUMMARY OF INVENTION

In a first aspect, the present invention provides a tissue ablation device comprising a sheath and a probe. The sheath is positionable within body tissue and comprises a distal end, a proximal end and a lumen extending therebetween. The probe comprises an elongate portion configured to be slidably received in the lumen, the elongate portion housing an electrode that is deployable from a distal end of the probe's elongate portion and into a substantially planar deployed configuration when the distal end of the elongate portion is located at or beyond the distal end of the sheath. The angle of deployment of the electrode from the distal end of the probe (and into the body tissue, in use) is selectable by orientating the probe with respect to the sheath.

The device of the present invention can advantageously be used to perform multiple ablations for each sheath insertion into body tissue, simply by changing the angle of deployment of the electrode into body tissue proximal to the sheath (i.e. by rotating the device's probe with respect to its sheath) between ablations. The combined effect of the multiple ablations has been found by the inventors to produce a volume of ablated tissue that is much greater than is possible using prior art devices having similar sized electrode configurations (i.e. without them being physically withdrawn and reinserted into the body tissue in a new location). As such, fewer electrodes (and/or smaller electrodes) are required in the ablation devices of the present invention which, in turn, enables thinner sheathes than those of currently available ablation devices to be used. As would be appreciated, the thinner the sheath of an ablation device, the less invasive the ablation procedure. Indeed, the inventors envisage that sheathes smaller than 2.0 mm (or even smaller than 1.5 mm) in cross sectional diameter will be able to be used in the present invention for the ablation of even very large tumours, enabling the procedure to be carried out percutaneously instead of laparoscopically or surgically. This is a reduction of over 25%, compared to commercially-available INCIRCLE devices (which have a diameter of 2.7 mm). As would also be appreciated, minimising the number of times ablation devices need to be inserted into a patient's body will also lead to simpler and less invasive procedures.

The present invention represents a significant divergence from conventional wisdom. As described throughout U.S. Pat. No. 9,060,782, for example, conventional wisdom in the art was that larger electrode arrays were required in order to ablate larger tumours. Indeed, the INCIRCLE devices described above have enjoyed significant commercial success for use in ablating relatively large tumours. The present inventor realised, however, that larger devices (specifically, the body piercing portions of the devices) were not compatible with minimally invasive procedures. Whilst surgeons may be qualified to insert probes having relatively large diameters into a patient's organs, such procedures would need to be performed at least laparoscopically or in intraoperative surgical procedures, and therefore need to be performed in an operating theatre. Ablation devices having smaller sheathes are known, but are only indicated for use in ablating small tumours and generally require that a grounding pad be used (with the attendant problems noted above). The unique configuration of the device invented by the inventors enables sheathes that are compatible with percutaneous insertion to be used, and the devices therefore operable by healthcare providers other than surgeons (e.g. interventional radiologists). Furthermore, (smaller) devices can be used to perform multi-step ablations that are no less effective than the ablations performable using the existing (larger) INCIRCLE devices.

Indeed, the inventors have found that two of the devices of the present invention can be operated in a manner whereby volumes of tissue much larger than that located between the devices' sheathes can be ablated, without having to reposition the sheathes. Ablation volumes extending well outwardly from a central zone between the devices' sheathes can be created by performing multiple ablations with the devices' electrodes deployed at different angles. Whilst “edge boosting” of ablations has been demonstrated previously, this was only possible in monopolar systems that required the use of earth pads and the attendant disadvantages.

In some embodiments, the probe may comprise a sheath abutting portion configured for receipt at the proximal end of the sheath when the distal of the probe's elongate portion is located at or beyond the distal end of the sheath (i.e. where the electrode can be deployed into tissue, in use).

In some embodiments, the sheath abutting portion of the probe and the proximal end of the sheath may comprise means (e.g. visual or tactile means) for indicating a relative orientation therebetween. The sheath abutting portion and the proximal end of the sheath may, for example, comprise surfaces that abut one another in use, the respective surfaces comprising indicia to visually show the relative orientation therebetween. Alternatively (or in addition), the sheath abutting portion and the proximal end of the sheath may, in some embodiments, comprise surfaces that abut one another in use, the respective surfaces comprising complimentary protrusions and recesses configured to mate when the sheath abutting portion and the proximal end of the sheath are orientated at predefined angles (e.g. about 0°, 90°, 180° and 270°).

In some embodiments, the electrode may bend (e.g. into a coil) upon deployment into its deployed configuration. The deployed configuration of the electrode may, for example be substantially circular in shape (e.g. having a diameter of 4 cm or less).

In some embodiments, the electrode may comprise a plurality of electrodes (e.g. 2 or 3 electrodes). Each of such electrodes may assume a similar or different configuration (e.g. being relatively larger or smaller than the others and/or having a different deployed shape to the others) in the substantially planar deployed configuration. In such embodiments, an electrode deployment configuration may be provided that provides a functionality (e.g. an ablation zone) not achievable by a single electrode. Each of the electrodes may, for example, be configured to be deployed independently of or concurrently with the other electrode(s). Each of the electrodes may, for example, be deployable through a respective orifice at the end of and/or along a side of the elongate portion at the distal end of the probe. As described below, such configurations of deployed electrodes can significantly affect the size and shape of the subsequent ablation.

In some embodiments, the probe for use in the ablation device of the present invention may be selectable from a plurality of available probes, with the electrodes in the available probes being configured to assume different (selectable) deployed configurations. In such embodiments, the operator can select probes having a deployed electrode configuration appropriate to their immediate needs, even mid-procedure after the sheath has been positioned within the patient's body tissue. For example, once the sheath is positioned with respect to a tumour, imaging could be used to determine a required size and shape of the deployed electrode. For example, if the sheath had been inserted slightly “off-centre”, a first relatively smaller electrode could be used to ablate part of the tumour and a second relatively larger electrode used to ablate the remainder of the tumour.

Such embodiments of the present invention provide the operator with an unprecedented degree of versatility in performing ablations, with a variety of electrodes being deployable through the lumen of the pre-placed sheath at a variety angles into the tissue surrounding a tumour.

In some embodiments, the ablation device may further comprise a deployment actuator which is operable to deploy the electrode from the distal end of the lumen. The deployment actuator may, for example, be operable to advance and retract the electrode between its deployed configuration and a retracted configuration.

In some embodiments, the ablation device may further comprise a handle that is coupleable to the probe and/or sheath. In some embodiments, the ablation device may further comprise a joining member for joining a first tissue ablation device to another tissue ablation device. The joining member may, for example, be configured to define a variable spacing between the joined tissue ablation devices.

In use, two of the ablation devices of the present invention may firstly be used together in order to define a central ablation zone between the devices' deployed electrodes, in a manner similar to that described in U.S. Pat. No. 9,060,782. Subsequently, however, and as will be described in further detail below, the devices' electrodes can be repeatedly deployed at a number of different angles with respect to the sheathes in order to ablate tissue around the edges of the central ablation zone and thereby produce a volume of ablated tissue that extends outwardly from the central zone. The method of the present invention can thus be used to produce ablations having a volume that was previously not thought possible with relatively small ablation devices.

In a second aspect therefore, the present invention provides a method for ablating tissue (e.g. containing a tumour) within an ablation zone in a patient's body (e.g. in a liver, spleen, kidney, lung, uterus or breast). The method comprises:
(a) positioning (e.g. percutaneously) the sheathes of two tissue ablation devices of the present invention in the patient (e.g. via the needle-wire-dilator-sheath procedure commonly used in radiological procedures and described in further detail below), with at least a portion of the ablation zone being located between the sheathes;
(b) orientating the probes of the tissue ablation devices with respect to the sheathes whereby the electrodes will deploy in a first configuration;
(c) deploying the electrodes in the first configuration and ablating tissue between the so-deployed electrodes to form a first ablated portion;
(d) retracting the electrodes back into the respective probes;
(e) reorientating the probes with respect to the sheathes whereby the electrodes will deploy in a second configuration;
(f) deploying the electrodes in the second configuration and ablating tissue between the so-deployed electrodes to form a second ablated portion;
(g) repeating steps (d) to (f) until the combined ablated portions define the ablation zone; and
(h) withdrawing the sheathes from the patient.

In another (less-favoured, although potentially useful for very small tumours e.g. thyroid) use, a single ablation device of the present invention may be used in order to define an ablation zone about the device's deployed electrode(s). In such uses, the device may either be bipolar or monopolar (which would require a grounding pad on the patient's skin). In a third aspect therefore, the present invention provides a method for ablating tissue within an ablation zone in a patient's body. The method comprises:
(a) positioning (e.g. percutaneously) the sheath of a tissue ablation device of the present invention in the patient (e.g. via the needle-wire-dilator-sheath procedure commonly used in radiological procedures and described in further detail below) at the ablation zone;
(b) orientating the probe of the tissue ablation device with respect to the sheath whereby the electrode will deploy in a first configuration;
(c) deploying the electrode in the first configuration and ablating tissue to form a first ablated portion;
(d) retracting the electrode back into the probe;
(e) reorientating the probe with respect to the sheath whereby the electrode will deploy in a second configuration;
(f) deploying the electrode in the second configuration and ablating tissue to form a second ablated portion;
(g) repeating steps (d) to (f) until the combined ablated portions define the ablation zone; and
(h) withdrawing the sheath from the patient.

As noted above, the multi-step ablation methods of the present invention enable a relatively small electrode (and hence a device having a relatively smaller sheath) to ablate relatively large tissue volumes. As such, minimally invasive techniques can be used to ablate tumours having a size which only the larger of the presently available RF ablation devices have conventionally been able to ablate.

In some embodiments, the angle between the first and second deployed configuration may be 180°. In effect, the electrodes are successively deployed in such embodiments on opposite sides of the sheath, which would usually result in the largest possible ablation zone from just two ablations. Such an ablation zone would be similar in volume to that produced in a single ablation using the ablation devices described in U.S. Pat. No. 9,060,782 which have electrode coils deployed on both sides of the trocar. Such devices, however, require the trocar to house six (or more) electrodes and therefore have a relatively large diameter (ca. 2.7 mm, or more), which may be incompatible with percutaneous procedures.

Furthermore, in some embodiments, the methods of the present invention may comprise three (or more) ablations. In an embodiment comprising three ablations, for example, the angle between the first and second deployed configurations may be 180° and the angle between the second and third deployed configurations may be 90°. As noted above, the first and second ablations would usually result in the largest possible ablation zone from just two ablations, and the third ablation would tend to enlarge the ablation zone due to the unconducive ablated tissue forcing the energy/heat around the periphery of and laterally to the combined first and second ablated portions. Such an “edge boost” enables the devices of the present invention to produce even larger ablations than prior art devices (having comparably sized electrodes).

In some embodiments, the methods may comprise an additional step of replacing the probe (or one or both of the probes in the method of the second aspect) with probe having a different electrode between ablations. The different electrode may, for example, differ in respect of one or more of its size and shape of its deployed configuration.

In some embodiments of the method of the third aspect, the ablation may occur between the deployed electrode and a ground plate (on the patient's skin). Notwithstanding the issues described above with mono-polar RF device ablations, the advantages provided by the present invention are also applicable to such systems and careful management of the ablation process may result in successful ablations.

In other embodiments of the method of the third aspect, the ablation device may be bipolar and ablation may occur between deployed electrodes of the device having an opposite polarity, or between the deployed electrode and a portion of the device (e.g. its sheath or probe) having an opposite polarity. Notwithstanding the issues noted above regarding the use of single ablation devices, the advantages provided by the present invention are also applicable to such systems and careful management of the ablation process may result in successful ablations.

In a fourth aspect, the present invention provides a method for ablating tissue (e.g. containing a tumour) within an ablation zone in a patient's body. The method comprises:
(a) positioning (e.g. percutaneously) the sheathes of a plurality (e.g. two or more) tissue ablation devices of the present invention in the patient, at least a portion of the ablation zone being located between the sheathes;
(b) orientating the probes of the tissue ablation devices with respect to the sheathes whereby the electrodes will deploy in a first configuration;
(c) deploying the electrodes in the first configuration and ablating tissue between the so-deployed electrodes to form a first ablated portion;
(d) retracting the electrodes back into the respective probes;
(e) reorientating the probes with respect to the sheathes whereby the electrodes will deploy in a second configuration;
(f) deploying the electrodes in the second configuration and ablating tissue between the so deployed electrodes to form a second ablated portion;
(g) repeating steps (d) to (f) until the combined ablated portions define the ablation zone; and
(h) withdrawing the sheathes from the patient.

Notwithstanding the benefits of minimally invasive procedures including those described above, the methods of the second, third and fourth aspects may involve positioning the sheathes of the tissue ablation device(s) of the present invention in the patient either laparoscopically or surgically.

In a fifth aspect, the present invention provides a bipolar tissue ablation method, wherein electrodes are repeatedly deployable in selectable orientations from pre-placed sheathes and operable to ablate previously unablated tissue therebetween, whereby successive ablations cumulatively grow the ablation.

Additional features and advantages of the various aspects of the present invention will be described below in the context of specific embodiments. It will be appreciated, however, that such additional features may have a more general applicability in the present invention than that described in the context of these specific embodiments.

### BRIEF DESCRIPTION OF DRAWINGS

Embodiments of the present invention will be described in further detail below with reference to the accompanying drawings, in which:

FIG. 1 shows a tissue ablation device in accordance with an embodiment of the present invention;

FIG. 2 shows the ablation device of FIG. 1 with its electrodes in a partially deployed configuration;

FIG. 3 shows a guidewire and needle for use in percutaneously inserting the device of FIG. 1 into a patient's body tissue;

FIG. 4 shows the guidewire of FIG. 3, over which a dilator and the sheath of the ablation device of FIG. 1 have been positioned;

FIG. 5 shows two of the ablation devices of FIG. 1 positioned in a patient's liver with the electrodes in a first deployed configuration;

FIG. 6 depicts the first ablation zone between the electrodes as deployed in FIG. 5;

FIG. 7 shows two of the ablation devices of FIG. 1 positioned in a patient's liver with the electrodes in a second deployed configuration;

FIG. 8 depicts the second ablation zone between the electrodes as deployed in FIG. 7, as well as the combined ablation zone;

FIG. 9 depicts a third ablation zone, which is produced when the electrodes are positioned in a third deployed configuration about half way between the first and second deployed configurations;

FIG. 10 depicts the ablation volumes achieved by performing successive ablations with electrodes deployed at angles of 0°, 180° and 90°;

FIG. 11 depicts the ablation volumes achieved by performing successive ablations with electrodes deployed at angles of 0°, 180°, 45/315° and 135/225°;

FIG. 12 shows the sheath and probe of an unassembled tissue ablation device in accordance with another embodiment of the present invention;

FIG. 13 shows the sheath and probe of FIG. 12 in an assembled configuration;

FIG. 14 shows an alternative mechanism for securing the probe to the sheath in a tissue ablation device in accordance with another embodiment of the present invention;

FIG. 15 depicts various deployed electrode configurations of two tissue ablation devices in accordance with another embodiment of the present invention positioned on either side of a tumour; and

FIG. 16 shows a tissue ablation device in accordance with an alternative embodiment of the present invention.

### DESCRIPTION OF EMBODIMENTS

As disclosed herein, the overarching purpose of the present invention is to ablate relatively large volumes of biological tissue using ablation devices that are physically smaller than those presently available. Due to their unique structure and functionality, the tissue ablation devices of the present invention can advantageously be operated to ablate volumes of tissue having a comparable size to that ablateable using conventional ablation devices.

As noted above, the present invention provides tissue ablation devices and methods for ablating tissue (e.g. containing a tumour) within an ablation zone (e.g. in a liver, spleen, kidney, uterus, lung or breast) in a patient's body. The tissue ablation device comprises a sheath and a probe. The sheath is positionable within body tissue and comprises a distal end (which, as described below, will be positioned in the body tissue in use), a proximal end (which, as described below, will be accessible by the device's operator in use) and a lumen extending therebetween. The probe comprises an elongate portion configured to be slidably received in the lumen and to house an electrode that is deployable from a distal end of the elongate portion of the probe and which, upon deployment and with the distal end of the elongate portion being located at or beyond the distal end of the sheath, assumes a substantially planar deployed configuration (which, as described below, will be positioned in the body tissue in use). The probe may also comprise a sheath abutting portion configured for receipt at the proximal end of the sheath when the distal end of the elongate portion is located at or beyond the distal end of the sheath. An angle of deployment of the electrode into the body tissue from the distal end of the probe is selectable by orientating the probe with respect to the sheath.

One method in accordance with the present invention comprises:
(a) positioning (e.g. percutaneously) the sheathes of two tissue ablation devices of the present invention in the patient (e.g. over a dilator which has been pre-placed using a conventional needle-wire-dilator approach used by interventional radiologists) such that at least a portion of the ablation zone is located substantially between the sheathes;
(b) orientating the devices' probes with respect to the sheathes such that the electrodes will deploy in a first configuration;
(c) deploying the electrodes in their first configurations and ablating tissue between the so-deployed electrodes to form a first ablated portion;
(d) retracting the electrodes back into their respective probes;
(e) reorientating the probes with respect to the sheathes such that the electrodes will deploy in a second configuration;
(f) deploying the electrodes in their second configurations and ablating tissue between the so-deployed electrodes to form a second ablated portion;
(g) repeating steps (d) to (f) until the combined ablated portions define the ablation zone; and
(h) withdrawing the sheathes from the patient.

Another method in accordance with the present invention comprises:
(a) positioning (e.g. percutaneously) the sheath of a tissue ablation device of the present invention in the patient at the ablation zone;
(b) orientating the device's probe with respect to the sheath such that the electrode will deploy in a first configuration;
(c) deploying the electrode in the first configuration and ablating tissue to form a first ablated portion;
(d) retracting the electrode back into the probe;
(e) reorientating the probe with respect to the sheath such that the electrode will deploy in a second configuration;
(f) deploying the electrode in the second configuration and ablating tissue to form a second ablated portion;
(g) repeating steps (d) to (f) until the combined ablated portions define the ablation zone; and
(h) withdrawing the sheath from the patient.

In the present invention, the tissue to be ablated may be any biological tissue susceptible to thermal coagulation. Typically, the biological tissue required to be ablated will comprise a tumour (usually a tumour which, due to its size, location or other characteristic is non-resectable). Tissue which may be ablated in accordance with the present invention includes, for example, uterine fibroids, liver tumours (benign or malignant), kidney tumours, lung tumours, brain tumours, thyroid tumours and breast tumours. Typically, the body tissue in which the sheath is positioned in use is an organ. The body tissue may for example, be a patient's liver, spleen, kidney, uterus, lung or breast.

As would be appreciated, tissue surrounding such tumours may also be ablated in use of the present invention. This may be advantageous because the outer portion of tumours can often be the most malignant and smaller tumours (which might not yet be detectable) may be spread out from the main tumour mass.

Ablation devices in accordance with the present invention may be used in percutaneous procedures, for example, to ablate tumours such as hepatocellular carcinoma (HCC), colorectal cancer hepatic metastases (CRCHM) and other liver metastases, gallbladder carcinoma, or hepatic adenoma (i.e. large-volume, symptomatic hepatic cavernous haemangiomas). Whilst some of the more significant advantages of the present invention relate to the ablation device's relatively small physical size (and hence its suitability for use in percutaneous procedures), persons skilled in the art would, however, appreciate that the devices and methods of the present invention are not limited to use solely in percutaneous procedures, and that the present invention also has application in procedures such as those carried out surgically or laparoscopically.

Although primarily intended for treatment of humans, it is envisaged that the present invention may also be used to treat similar conditions in non-human animals.

The general principals of operation and advantages of RF ablation devices such as those of the present invention and their use in an ablating configuration on either side of a target area of tissue (i.e. one containing a tumour) are comprehensively described in U.S. Pat. No. 9,060,782. In brief, accurate device placement (specifically the devices' sheathes) may be facilitated with an ultrasound guidance tool (for example) that allows the use of ultrasound to directly visualize the target area to produce optimal or near-optimal ablations. Using such a technique, the sheathes of two ablation devices (for example) could be positioned in a patient's body tissue on opposing sides of the target area. Unlike conventional monopolar ablation systems, the positioning of sheathes in the patient would typically avoid tumour contact at all stages in the procedure, thereby minimizing or avoiding the risk of tumour seeding. Furthermore, embodiments of the devices described herein, as a result of their multi-device and bipolar configuration in use, do not require return electrodes or grounding pads, and therefore have more efficient energy distribution at the tumour site so lower power settings can be used (i.e. in comparison with conventional monopolar RF systems). This allows for safer procedures with lower power settings, no grounding pads and no skin burns.

The interface between the electrode surface and the tissue in RF ablation is analogous to a fuse, or “fusible link”. The electrode(s) of the device(s) is/are configured to “overlay” the target tissue area so that the ablation procedure progresses from the outside to the inside of the target tissue area, between the devices' deployed electrodes. The electrode configuration increases the amount of tissue surface area that can be engaged by the devices because a larger amount of tissue is “enclosed” by the electrodes when compared to a conventional monopolar device (which places the electrode at or near the centre of the target tissue area). This configuration, in effect, provides a larger “fuse” for receiving the applied energy, thus allowing for the delivery of more energy (current), along with a relatively slower time constant or ramp of the increase in impedance as the procedure progresses.

Embodiments of the devices of the present invention can overcome numerous issues associated with the use of conventional monopolar RF ablation devices, due to their “outside-to-inside” heating and, consequently, high energy transfer to the target tissue. The high energy transfer allows the devices to overcome larger heat sinks (e.g. blood vessels), while a defined energy envelope controls potential runaway by keeping the energy confined to the targeted area. This allows substantially all of the delivered energy to go into the target area, instead of radiating outwardly. The device configuration can also provide a more uniform energy density, with the energy being delivered to the critical outer surface of a tumour first, and with a high energy density. The energy produced by the electrodes passes through the target tissue as it passes between the electrodes, and this produces and maintains a more uniform energy density relative to conventional devices. End point measurements of impedance are also more reliable since virtually everything being measured is the targeted tissue itself. This combination of high energy delivery to overcome heat sinks, energy delivery at the surface of the target tissue volume, energy focused only into the target area, as well as a high and more uniform energy density helps the devices of an embodiment to produce faster, more uniform, and more repeatable ablations.

The electrode of the devices of the present invention needs to be electrically connected to an energy source in order for ablation to occur. Suitable energy sources are known in the art and some are described in more detail in U.S. Pat. No. 9,060,782, for example. Such an energy source may be provided in the form of an electrical generator, which can deliver pre-specified amounts of energy at selectable frequencies in order to ablate tissue. The energy source may include at least one of a variety of energy sources, including electrical generators operating within the radio frequency (RF) range. More specifically, and by way of example only, the energy source may include a RF generator operating in a frequency range of approximately 375 to 650 kHz (e.g. 400 kHz to 550 kHz) and at a current of approximately 0.1 to 5 Amps (e.g. of approximately 0.5 to 4 Amps) and an impedance of approximately 5 to 100 ohms. As would be appreciated, variations in the choice of electrical output parameters from the energy source to monitor or control the tissue ablation process may vary widely depending on tissue type, operator experience, technique, and/or preference.

The tissue ablation device of the present invention includes a sheath that is configured to be positioned in a patient's body tissue using conventional techniques, examples of which will be described below. The sheath includes a distal end that, in use, is positioned in a patient's body tissue at the site to be ablated, a proximal end that, in use, is accessible to the device's operator and a lumen extending therebetween.

As noted above, due to the devices of the present invention having to contain only one electrode (or one set of electrodes) that deploy into their substantially planar configuration, instead of the plurality of electrodes/electrode sets which deploy from both sides of the sheath into the electrode arrays described in U.S. Pat. No. 9,060,782, for example, then the devices' sheathes may be up to about half as thin as the sheathes of conventional ablation devices. Indeed, the inventors have found that sheathes having a diameter of significantly less than 2.5 mm (e.g. less than about 2.2 mm, less than about 2.0 mm, less than about 1.8 mm, less than about 1.6 mm, less than about 1.5 mm, less than about 1.3 mm, less than about 1.2 mm or even less than about 1.0 mm). are effective. Sheathes carrying only one electrode may be even thinner. The sheath may have any suitable length, depending on the location of the body tissue to be ablated in the patient.

The sheath may be formed from any material compatible with its use for its intended purpose. Typically, the sheath would be formed from metallic materials such as stainless steel or nickel titanium alloys, although plastic materials including Ultem, polycarbonate, and liquid crystal polymer might also be used.

The distal end of the sheath may have a configuration that enables it to penetrate tissue (e.g. like a trocar, for example) or may be non-tissue penetrating. Given that the procedures for which the device of the present invention will be indicated for use in are mainly percutaneous and to be carried out by interventional radiologists (for example), the distal end of the sheath need not be tissue-penetrating, as it will likely be inserted using a needle-wire-dilator-sheath approach, discussed in further detail below.

The proximal end of the sheath may take any form that provides access to the lumen. In the simplest of embodiments, the proximal end of the sheath may simply comprise an aperture defining a proximal end of the lumen, and into which may be inserted the probe's elongate portion. In other embodiments, however, the proximal end of the sheath would typically be configured in order to improve the handleability of the sheath and to provide for user-friendly and beneficial interactions with the probe. The proximal end of the sheath may, for example, include a body having a complimentary shape to that of the probe's sheath abutting portion. The proximal end of the sheath may, for example, include a guide portion for more easily aligning the elongate portion of the probe with respect to the sheath's lumen.

The tissue ablation device of the present invention also includes a probe. The probe includes an elongate portion and, optionally, a sheath abutting portion. The elongate portion is configured to be slidably received in (i.e. through) the lumen, typically in a relatively snug manner. The rotatability of the probe's elongate portion within the sheath (i.e. pre-deployment of the electrodes) is key to the functionality of the ablation device of the present invention, and any structure of the probe and sheath needs to not unduly restrict such rotation.

The probe's elongate portion has a length the same as, or slightly longer than, that of the sheath such that, once the sheath and probe are appropriately configured, the distal end of the elongate portion is located at or beyond the distal end of the sheath. Advancement of the probe too far beyond the distal end of the sheath would typically be limited (e.g. physically, e.g. by the sheath abutting portion) in order to ensure patient safety and precision in use of the device. The respective positions of the distal ends of the probe and sheath will depend on how the electrode(s) deploy, as will be discussed in further detail below.

It should be noted that, in embodiments where the probe extends outwardly from the distal end of the sheath, this would usually be into body tissue that had been pre-dilated (e.g. during insertion into and positioning of the sheath within the body tissue). The distal end of the probe's elongate portion would not usually be configured to be tissue piercing, although could be, should there be advantages of doing so.

The elongate portion of the probe houses an electrode (or electrodes) that is deployable from the probe's distal end and which, upon deployment, assumes a substantially planar deployed configuration. An angle of deployment of the electrode from the distal end of the probe is selectable by orientating the probe with respect to the sheath, as will be described in further detail below.

The electrode (or electrodes, where multiple electrodes are provided, for example) may be housed in the elongate portion of the probe in any suitable manner, provided that it is capable of achieving the functionality disclosed herein. Typically, the electrode(s) will be housed in the probe's elongate portion's lumen, although a proximal portion of the electrode(s) (i.e. that is not deployed) may extend out from the probe and into a handle of the device, for example. The electrode(s) may be deployed from the very end of the probe's elongate portion. Alternatively (or in addition), the elongate portion may have an orifice or aperture or a plurality of orifices/apertures arranged along a side thereof and through which the electrode(s) are deployable.

The deployed electrode delivers RF energy to the tissue to be ablated, and may have any configuration which is compatible with this functionality and which is not incompatible with other components of the device. The electrode may have many different sizes (including lengths and widths/thicknesses), depending upon the energy delivery parameters (current, impedance, etc.) of the corresponding system. The use of electrodes having different thicknesses may, for example, enable the energy/energy density in the target tissue to be controlled. In some embodiments, for example, the electrode may have a thickness in the range of about 0.5 mm to about 1.5 mm (e.g. a thickness of about 0.5 mm, about 0.6 mm, about 0.7 mm, about 0.8 mm, about 0.9 mm, about 1.0 mm, about 1.1 mm, about 1.2 mm, about 1.3 mm, about 1.4 mm or about 1.5 mm). Electrodes thinner than about 0.5 mm may not be able to carry an appropriate amount of current and may be susceptible to breakage, whilst electrodes thicker than about 1.5 mm would require probes/shafts having a corresponding diameter.

The electrodes may have any deployed length sufficient to generate or create an ablation diameter approximately in the range of about lcm to about 7 cm, but are not so limited. The spacing between the electrodes of two (or more) devices positioned in a patient's body tissue can also be used to control the energy density.

The electrodes may be formed from any electrically-conductive material, although they may also include non-conducting materials, coatings, and/or coverings in various segments and/or proportions, provided that such are compatible with the energy delivery requirements of the corresponding procedure and/or the type of target tissue. Examples of materials which may be used to form the electrodes of the present invention include stainless steel, carbon steel or nickel-titanium alloys, such as those sold as “Nitinol Wire” by Fort Wayne Metals. It should also be noted that electrodes which are not intended to perform multiple ablations may be capable of being formed from lighter materials, or materials otherwise not suitable for multiple reuses.

The electrode may take any suitable form, such as a flat wire electrode, a round wire electrode, a flat tube electrode or a round tube electrode. As will be appreciated, such electrodes would produce different energy profiles for ablation of selected tissue types, etc.

Typically, an end of the electrode is adapted for piercing body tissue (i.e. during its deployment), for example by being sharpened. In some embodiments, however, a tissue piercing functionality may not be required, for example, where this is performed during insertion and positioning of the sheath (e.g. the dilator may be “over inserted” into the tissue and them withdrawn slightly in order to provide pre-dilated tissue into which the electrode can be deployed).

The electrode is configured to assume a deployed configuration upon its deployment from the distal end of the probe. The electrode may, for example, bend upon deployment into its deployed configuration. In such embodiments, the electrodes may include or be formed from materials that support bending and/or shaping of the electrodes post-deployment. The electrodes may, for example, include pre-bent wire (e.g. Nitinol, as described above) which, once deployed from the confines of the probe's lumen, is free to assume its bent configuration.

The deployed configuration of the electrode may take any form compatible with ablation of body tissue proximal to the electrode. Typically, the electrode bends into a coil upon deployment into its deployed configuration, this being something readily achievable using conventional electrodes and devices, such as those described in U.S. Pat. No. 9,060,782.

The deployed configuration of the electrode may, for example, be substantially circular in shape. Alternatively (or additionally, in embodiments where the electrode comprises a plurality of electrodes), the electrode may assume an elliptical shape once deployed. In some embodiments, it may be advantageous to only partly deploy the electrode(s) (e.g. if only a very small ablation is necessary). The electrode configuration or geometry also makes use of electrode “rings”, which have the effect of “long” electrodes having a large surface area and therefore large tissue engagement area. Thus, the result of the combination of electrode surface area, individual electrode spacing, and overall device configuration or geometry is complete ablations.

The deployed configuration of the electrode may have any suitable size, bearing in mind the overarching requirement that the device is primarily intended for percutaneous operation and therefore that fewer electrodes and/or smaller electrode are generally preferred. In some embodiments, for example, the deployed configuration of a generally-circularly-shaped electrode may have a diameter of 2.5 cm or less (e.g. 2 cm or less, 1.5 cm or less, lcm or less or 0.5 cm or less). The inventors have demonstrated that ablations of up to about 7 cm are achievable using two ablation devices of the present invention having sheaths with a diameter of 1.6 mm (around 25% smaller than the sheaths of commercially available tissue ablating devices) positioned about 4 cm apart and having three 2 cm electrodes deployed from one side of each probe. For ablation of very small lesions (e.g. in the thyroid), however, devices with one electrode having a coil diameter of 0.5 mm may be suitable.

It is within the ability of a person skilled in the art, based on the teachings contained herein and in U.S. Pat. No. 9,060,782 to determine an appropriate electrode for use in the device of the present invention for any given ablation procedure.

In some embodiments, the electrode may comprise a single electrode which assumes its deployed configuration post-deployment. In other embodiments, however, the electrode may comprise a plurality of electrodes (e.g. 2, 3 or 4 electrodes). Each of such electrodes may assume the same or a different configuration upon deployment. Such embodiments may be beneficial in ablating relatively larger, or uneven shaped, tumours, for example, where electrodes having a composite shape are better able to ablate the tumour (e.g. because of a shape of the composite deployed electrode and/or an intensity of the RF energy applied by the electrodes). In some embodiments, the plurality of electrodes may be configured to assume deployed configurations having different sizes and/or shapes. In some embodiments the plurality of electrodes may be configured to assume deployed configurations offset to one another (e.g. along a length of the distal end of the probe), thus providing a greater ablating surface area.

The electrodes in such embodiments of the present invention may be electrically connected to or insulated from each other, and may have the same or different polarity to each other. The number of electrodes in such embodiments is limited only by the functional requirements of and the overarching purpose of the present invention, namely that the electrodes are deployable from the probe and that the ablation devices are generally smaller than those disclosed in U.S. Pat. No. 9,060,782, for example.

The electrode(s) assume a substantially planar deployed configuration upon deployment. Thus, a plane is defined by the deployed electrode(s), the orientation of which is controllable by the operator simply by orientating the probe with respect to the sheath. In embodiments where the device includes two or more electrodes, each of the electrodes should ideally deploy in about the same plane, or the degree of control of the ablation procedure may be lost. Relatively small deviations from planarity may be appropriate in some applications and embodiments.

In some embodiments, the same electrode or electrodes may be used for each ablation in multi-step ablations in accordance with the present invention. In other embodiments, however, it may be advantageous to use different electrodes during the multi-step ablation, with the electrodes being selectable from a number of available electrodes that are configured to assume different deployed configurations. Typically, for practical reasons (handling pre-bent and sharpened electrodes may, for example, be challenging), it would likely be the probe that would be selectable from a number of different probes, each of such probes having electrodes configured to assume selectively deployed configurations.

For example, tumours often have an irregular shape and, no matter how carefully the devices' sheathes (or the device's sheath) are placed on opposing sides of the tumour, it is likely that an ablation to one side of the so-positioned sheaths will need to be larger than that to the opposite side of the sheathes. In such embodiments, for example, first probes (which may be the same or different) may be inserted into the lumens of the appropriately positioned sheathes and their electrode deployed and operated to ablate the side of the tumour therebetween. The electrodes may then be retracted back into their respective probes and the probes withdrawn completely from their respective sheathes. Second probes (which may be the same or different), having electrodes that are larger/smaller/configured to assume a different deployment configuration, etc. are then inserted into the lumens of the sheathes in an opposite orientation to that of the first probes and their electrodes deployed and operated to ablate the other side of the tumour.

In this manner, the operator of the device has an unprecedented versatility for treating a tumour during a procedure (even should a sheath have been incorrectly placed). As would be appreciated, it is often only during such procedures that the physical characteristics of the tumour are discovered (noting that tumours may not always be spherical). The method of the present invention allows for a more tailored ablation regimen than has previously been possible without requiring the device to be reinserted multiple times.

As noted above, an angle of deployment of the electrode from the distal end of the probe is selectable by orientating the probe with respect to the sheath. In this manner and as will be described in further detail below, smaller devices having smaller electrodes can be used to ablate relatively large volumes of tissue.

In some embodiments, the probe further comprises a sheath abutting portion configured for receipt at the proximal end of the sheath when the distal end of the elongate portion is located at or beyond the distal end of the sheath. Such a feature provides a physical indicator of the probe's distal end being in a positon for deployment of the electrode, as well as the other advantages described herein.

In some embodiments, the sheath abutting portion of the probe and the proximal end of the sheath may comprise means for indicating a relative orientation therebetween. Such means may help an operator to ensure that a desired ablation pattern is achieved, notwithstanding not being able to physically see the deployed electrodes. The sheath abutting portion and the proximal end of the sheath may, for example, comprise visual or tactile means for indicating a relative alignment therebetween.

In one such embodiment, the sheath abutting portion and the proximal end of the sheath may comprise surfaces that abut one another in use, the respective surfaces comprising indicia (e.g. markings on the sheath and probe which visibly contrast with the other surfaces) to visually show a relative orientation therebetween. Alignment of relevant indicia on the probe and sheath can then readily be achieved during the procedure. The indicia may include angle markings, e.g. 0°, ±45°, ±90°, ±135° and 180°, or 0°, 45°, 90°, 135°, 180°, 225°, 270° and 315° or just 0°, 90°, 180° and 270°, for example, which correspond with the angle of deployment of the electrode(s) from the probe.

In another such embodiment, the sheath abutting portion of the probe and the proximal end of the sheath may comprise surfaces that abut one another in use, where the respective surfaces comprise complimentary protrusions and recesses configured to mate when the sheath abutting portion and the proximal end of the sheath are aligned at predefined angles. Correct alignment of the probe (electrode) and sheath can then be achieved by “feel”. As would be appreciated, a combination of visual and tactile means for indicating a relative alignment between the sheath abutting portion and the proximal end of the sheath might also be advantageous.

Any relative alignment between the sheath abutting portion and the proximal end of the sheath (and hence the angle of deployment of the electrode(s) into the body tissue) may be marked on the sheath and/or probe. Due to space constraints, however, only a few such angles would likely be shown. For example, predefined angles of 0°, 90°, 180° and 270° may be included, these being the angles of deployment most likely to be routinely used. In some embodiments, a line marker or otherwise may be included in order to indicate 45°, 135°, 225° and 315°.

Typically, the probe and/or sheath would also include a locking mechanism in order to ensure that, once selected by the operator, the orientation of the probe with respect to the sheath remains fixed.

The tissue ablation device of the present invention will also require other components in order for it to be used to ablate tissue. Some of these components are described below, whilst others are described in U.S. Pat. No. 9,060,782.

In some embodiments, the tissue ablation device may include a deployment actuator (or handle, plunger, switch, button, etc.) which is operable to deploy the electrode from the distal end of the probe. The deployment actuator may be manually operable, for example, to advance and retract the electrode between its deployed and retracted configurations.

In some embodiments, the tissue ablation device may include a handle that is coupleable to the probe and/or sheath. Such a handle may be ergonomically configured to enable an operator to manipulate the device in the required manner, both to insert the shaft/probe into the tissue and to deploy/retract the electrodes, etc.

In some embodiments, the tissue ablation device may include a joining member for joining a first tissue ablation device to another tissue ablation device. In this manner, two devices may be operated at the same time by an operator. In some embodiments, the joining member may be configured to define a variable spacing between the joined tissue ablation devices in order for the devices' sheathes to be inserted in the appropriate alignment on opposing sides of a tumour, for example.

The components of the tissue ablation devices of the present invention may be made from conventional materials, such as those described in U.S. Pat. No. 9,060,782.

As noted above, the present invention also provides methods for ablating tissue within an ablation zone in a patient's body. In a first method, two of the tissue ablation devices of the present invention are used. The first method comprises the following steps:
(a) positioning (e.g. percutaneously positioning) the sheathes of two of the tissue ablation devices in the patient, at least a portion of the ablation zone being located between the sheathes;
(b) orientating the devices' probes with respect to the sheathes such that the electrodes will deploy in a first configuration;
(c) deploying the electrodes in the first configuration and ablating tissue between the so-deployed electrodes to form a first ablated portion;
(d) retracting the electrodes back into the respective probes;
(e) reorientating the probes with respect to the sheathes such that the electrodes will deploy in a second configuration;
(f) deploying the electrodes in the second configuration and ablating tissue between the so-deployed electrodes to form a second ablated portion;
(g) repeating steps (d) to (f) until the combined ablated portions define the ablation zone; and
(h) withdrawing the sheathes from the patient.

In a second method, only one tissue ablation device of the present invention is used. The second method comprises the following steps:
(a) positioning (e.g. percutaneously positioning) the sheath of a tissue ablation device in the patient at the ablation zone;
(b) orientating the device's probe with respect to the sheath such that the electrode will deploy in a first configuration;
(c) deploying the electrode in the first configuration and ablating tissue to form a first ablated portion;
(d) retracting the electrode back into the probe;
(e) reorientating the probe with respect to the sheath such that the electrode will deploy in a second configuration;
(f) deploying the electrode in the second configuration and ablating tissue to form a second ablated portion;
(g) repeating steps (d) to (f) until the combined ablated portions define the ablation zone; and
(h) withdrawing the sheath from the patient.

In some embodiments of the second method, only one electrode is located at the ablation zone and ablation is caused to occur between the deployed electrode and a return electrode, which may be a ground plate on the patient's skin. As will be appreciated, such embodiments of the second method are monopolar ablation systems and may not have all of the advantages of the multi-device, bipolar ablation systems described herein. However, the inventors believe that some of the advantages associated with the device's smaller sheath and single insertion, multi-step ablation method of the present invention are also relevant to the second method.

In some embodiments of the second method, the ablation device may itself be bipolar and ablation may, for example, occur between deployed electrodes of the device having an opposite polarity or between the deployed electrode and a portion of the probe having an opposite polarity. Notwithstanding the issues noted above regarding the use of single ablation devices, the advantages provided by the present invention may also be applicable to such systems and careful management of the ablation process may result in successful ablations.

In some embodiments, the angle between the first and second configurations of the deployed electrodes may be about 180°, which provides the widest possible ablation zone. As noted above, the electrodes are deployed in such embodiments on substantially opposite sides of the sheath, which results in the widest possible ablation zone from just two ablations. Such an ablation zone would be similar in volume to that produced in a single ablation using the ablation devices described in U.S. Pat. No. 9,060,782, which have electrode coils deployed on both sides of the trocar, but using a thinner device and one that is especially compatible for use in percutaneous procedures.

Ablations to either side of the sheathes would usually be those conducted first and second, and would result in a central ablated zone which encompasses a majority of the target tissue (e.g. a tumour). This ablated tissue will no longer conduct electricity, and any further ablations carried out with the electrodes deployed laterally (i.e. facing generally away from the central ablated zone) will force the applied energy around the central ablation, causing a lateral extension to and enlargement of the ablation.

In some embodiments therefore, the method may comprise three or more ablations, with these subsequent ablations potentially resulting in even larger volumes of ablated tissue and/or ablated volumes of tissue having shapes responsive to the location of the target zone. For example, tumours may be located towards an edge of a body organ such as a liver or close to a blood vessel and it would not be beneficial (and may be extremely dangerous) to deploy the electrodes outside of the liver or into the vessel.

In some embodiments, the angle between the first and second configuration may, for example, be about 180° and the angle between the second and third configuration maybe about 90°. Such an ablation method can, as will be described in more detail below, be used to produce a relatively large ablation zone (especially when compared to the relative size of devices' sheathes and their deployed electrodes).

In some embodiments, the method may comprise four ablations, carried out with the electrodes deployed at 0° and 180° and then at either +/−90° or +/−45°/135°. Choosing between deployment angles of +/−90°/270° or +/−45°/135° for the 3^{rd}/4^{th} ablations may depend on factors such as tumour size and location, for example. If a tumour is close to the edge of the liver or a blood vessel, for example, doing a 90°/270° ablation might deploy the probes outside of the liver or into the vessel, etc. In such circumstances, choosing a “closer” 45°/135° ablation (see the discussion below) may be more appropriate.

Such an ablation method can, as will be described in more detail below, be used to produce a relatively large ablation zone (especially when compared to the relative side of the deployed electrode). As would be appreciated, the ablation devices and methods of the present invention provide for a unique bipolar “edge boosted” ablation, previously uncontemplated in bipolar systems and without the use of earth pads.

In some embodiments and for the reasons and advantages discussed above, the methods may comprise the additional step of replacing the probe with a probe having a different electrode between ablations. As previously, the different electrodes may differ in respect of the size and/or shape of its deployed configuration.

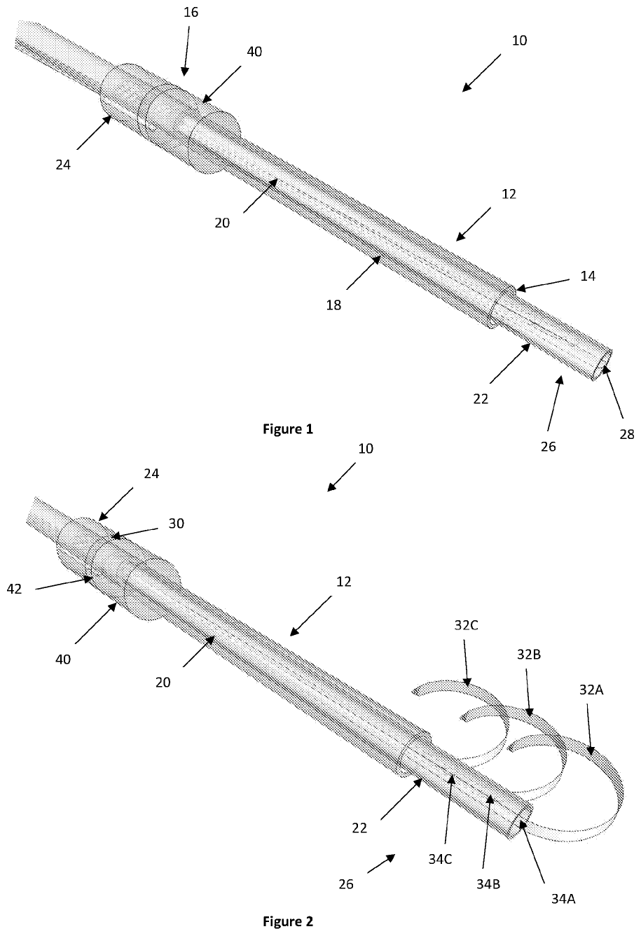

Specific embodiments of tissue ablation devices and ablation methods in accordance with the present invention will now be described, by way of example only, with reference to the drawings. Referring firstly to FIGS. 1 and 2, a tissue ablation device in the form of ablation device 10 is shown. Device 10 has a sheath 12 and probe 20 (the sheath 12 is shown as being translucent in FIGS. 1 and 2 so that the probe 20 can be seen). Sheath 12 has a distal end 14 which, in use and as described below, would be positioned in the body tissue (e.g. liver) of a patient. Sheath 12 also has a proximal end 16 (see also FIG. 4) and a lumen 18 that extends between the distal 14 and proximal 16 ends. A sheath cap 40 is either fixed to or integrally formed at the proximal end 16 of the sheath 12 and has an outwardly facing (in use) annular surface 42.

Probe 20 has an elongate portion in the form of sleeve 22, which is sized and shaped to be snugly received within lumen 18, and a sheath abutting portion 24. Probe 20 also has a distal end 26 (located at the distal end of sleeve 22 to the sheath abutting portion 24), and a lumen 28 that extends through the sleeve 22. Sheath abutting portion 24 has an inwardly facing (in use) annular surface 30, which extends annularly around the sleeve 22.

Device 10 also includes an electrode, shown in the form of a plurality of flat wire electrodes 32A, 32B and 32C (collectively referred to herein as electrodes 32). The electrodes 32 are housed within the lumen 28 of sleeve 22 until they are caused to be deployed in the manner described below. Although not shown, the electrodes 32 would be electrically connected to a source of energy such that, once deployed and connected to the source of energy, they can ablate tissue in the manner described herein.

In the assembled configuration shown in FIGS. 1 and 2, the probe's sleeve 22 is positioned within the sheath's lumen 18, within which it can freely rotate, and the sheath abutting portion 24 is proximal to the sheath cap 40 (and hence the sheath's proximal end 16). In this configuration, the inwardly facing surface 30 (i.e. facing towards the body tissue, in use) of the probe's sheath abutting portion 24 is brought to bear against the outwardly facing surface 42 (i.e. facing away from the body tissue, in use) of sheath cap 40.

As can be seen in FIGS. 1 and 2, the distal end 26 of probe 20 projects outwardly from the distal end 14 of the sheath 12 when surfaces 30 and 42 bear against one another. In this configuration, apertures 34A, 34B and 34C of the sleeve 22 are exposed. Aperture 34A is provided at the tip of sleeve 22, whilst apertures 34B and 34C are provided in line along the side wall of the sleeve. In this manner, the electrodes 32A, 32B and 32C housed within sleeve 22 are deployable in the in-line manner as described below between the fully retracted position shown in FIG. 1 and the partially deployed configuration shown in FIG. 2. The in-line overlapping electrode coils 32 define an electrode array capable of ablating body tissue in the manner described in U.S. Pat. No. 9,060,782.

In this embodiment, electrodes 32 are formed from pre-bent flat wire and, as such, assume a coiled configuration (having a diameter of about 3 cm) upon deployment. As can be seen, the ends of the electrodes 32 are sharpened, which assists with tissue penetration. Once in their deployed configuration, ablation may be performed by supplying appropriate energy to the electrodes 32 (e.g. via electrical wires extending between the device 10 and a power source, not shown).

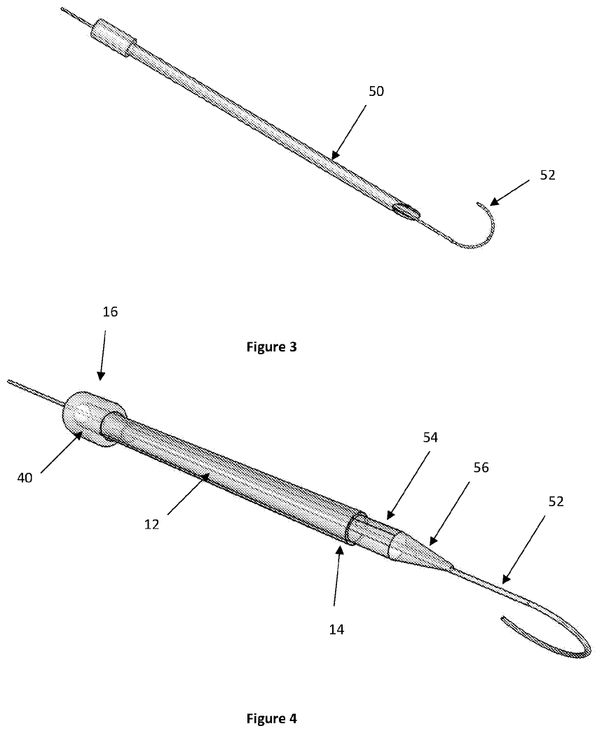

Use of deice 10 in performing a multi-step ablation procedure in accordance with an embodiment of the present invention will now be described with reference to FIGS. 3 to 9. FIGS. 3 and 4 relate to the method for positioning the sheath 12 within the patient's body tissue, whist FIGS. 5 to 9 relate to the ablation stages of the procedure. For convenience, the procedure described below will be described in the context of ablating a tumour in a patient's liver, although it will be appreciated that the procedures described below could readily be adapted by a person skilled in the art to treat other tumours in other body tissues.

The sheath 12 may be positioned within the patient's liver using any conventional technique. One such technique that is routinely used by interventional radiologists in percutaneous procedures is the so-called “needle-wire-dilator-sheath” procedure. Referring firstly to FIG. 3, a needle 50 having an appropriate gauge is carefully inserted through the patient's skin and into their liver, and advanced into a location relative to a tumour to be treated. Typically, the needle will be inserted close to, but not into, the tumour in order to eliminate the possibility of the tumour seeding complications noted above from occurring. Visualisation techniques could, for example, be employed in order to appropriately positon the needle. As the needle has a fine gauge and is relatively easy to control, it is unlikely that the operator might accidentally mis-position the needle, with the attendant consequences. Once needle 50 is appropriately positioned, a wire 52 is passed through the needle's lumen in order to define its track, and the needle 50 is then removed.

A dilator 54 having a tissue dilating point 56 and a lumen 58 is then used to dilate the tissue along the track left by the needle 50. The opposite end of wire 52 (i.e. the end outside of the patient's body) is fed through the lumen 58 and the sheath 12 is positioned over the dilator 54 before the dilator (and hence the sheath 12 carried by the dilator) is inserted into the patient. Advancing the dilator 54 along the tack left by the needle 50, as guided by wire 52, dilates the tissue around the needle track. Once the dilator 54 (or, more appropriately, the sheath 12) is in an appropriate position (visualisation techniques can again be used to determine this), the dilator 54 and the wire 52 can both be withdrawn from the patient, leaving the sheath positioned within the patient's liver proximal to the tumour. If required (e.g. to dilate tissue for the distal end 26 of the probe 20), the dilator 54 could be advanced slightly further into the patient's liver before it is withdrawn. A second sheath 12 would subsequently be positioned in the patient's liver on the other side of the tumour using the same technique.

Once so-positioned, the sheathes 12, 12 remain in the same location throughout the entirety of the multi-step ablation procedure. As would be appreciated, this is a much simpler and safer procedure than those which require multiple injections.

Referring now to FIG. 5, shown is a patient's liver 60 into which two devices 10, 10 are positioned on either side of a tumour 62. The probes 20, 20 have been orientated in the sheaths' lumens 18, 18 in a first respective orientation (of the probe with respect to the sheath, the sheath being effectively in a fixed position due to it being in the patient's liver), which is defined to be 0 degrees. The sleeves 22, 22 have been advanced through the lumens 18, 18 and their distal ends 26, 26 project out from the sheaths' distal ends 14, 14 and into pre-dilated portions of the liver 60. Movement of each sleeve 22 further into the liver 60 is prevented due to the inwardly facing surface 30 of the sheath abutting portion 24 and the outwardly facing surface 42 of the sheath cap 40 abutting one another. The orientation of each probe 20 in its respective sheath 12 can be fixed using the mechanism described below.

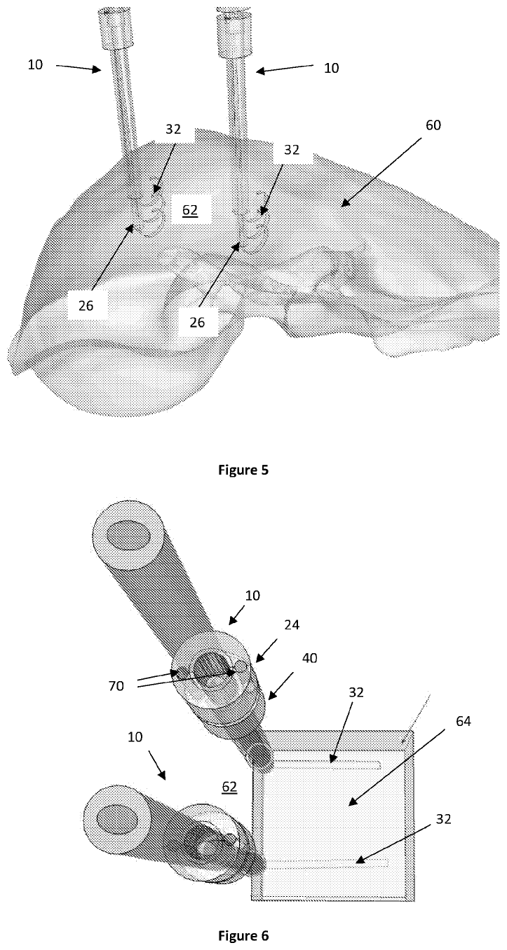

The electrodes 32 of each device 10 have been mostly deployed (a complete coil would be formed by each electrode upon full deployment) in the first configuration shown in FIG. 5 (and schematically depicted in FIG. 6). The combined electrodes 32A, 32B and 32C overlap in their deployed configurations, effectively defining planar and generally rectangular electrode arrays extending from one side of the probes 20, 20 and having a height about twice that of its width. Tissue in the liver 60 located between the combined electrodes 32, 32 of the devices 20, 20 will be ablated upon application of an appropriate source of energy to the electrodes in a conventional manner.

Tumour 62 is, in this embodiment, larger than would be ablateable using the devices 10 in the configuration shown in FIG. 5. Using conventional ablation devices and techniques, it would have been necessary to use a larger ablation device, of the kind described in U.S. Pat. No. 9,060,782 for example, or to perform a number of ablations from a number of different locations (necessitating the ablation device to be inserted into the patient's liver a corresponding number of times). As noted above, whist clinically effective, such conventional procedures have associated drawbacks. The multi-step ablation method of the present invention, however, enables smaller devices such as device 10, having correspondingly smaller shafts 12 and electrode arrays 32 to be used in multi-step procedures to ablate even relatively large tumours, such as tumour 62.

Referring now to FIG. 6, which is an illustrative view looking down onto the liver 60 along the length of the devices 10, 10, with some of each device's components being depicted as being translucent so that other components can be seen, a first ablation zone 64 is shown between the deployed electrodes 32, 32. FIG. 6 also shows the upper surfaces of electrodes 32, 32 whist in their first deployed configuration, as described above in relation to FIG. 5. As can be seen, sheath abutting portion 24 abuts sheath cap 40 and, in this embodiment, these components are effectively locked into a fixed orientation with respect to one another via pin and recess type couplings 70, 70 located on opposite sides of the portion 24 and cap 40.

Upon application of an appropriate amount of energy and for an appropriate amount of time, tissue in the first ablation zone 64 is heated from the outside-in (i.e. starting from the electrodes 32, 32 and working towards a mid-point between them) to a temperature at which the tissue is completely ablated. As can be seen from FIG. 6, some ablation of tissue surrounding first ablation zone 64 may also occur, but to a lesser extent.

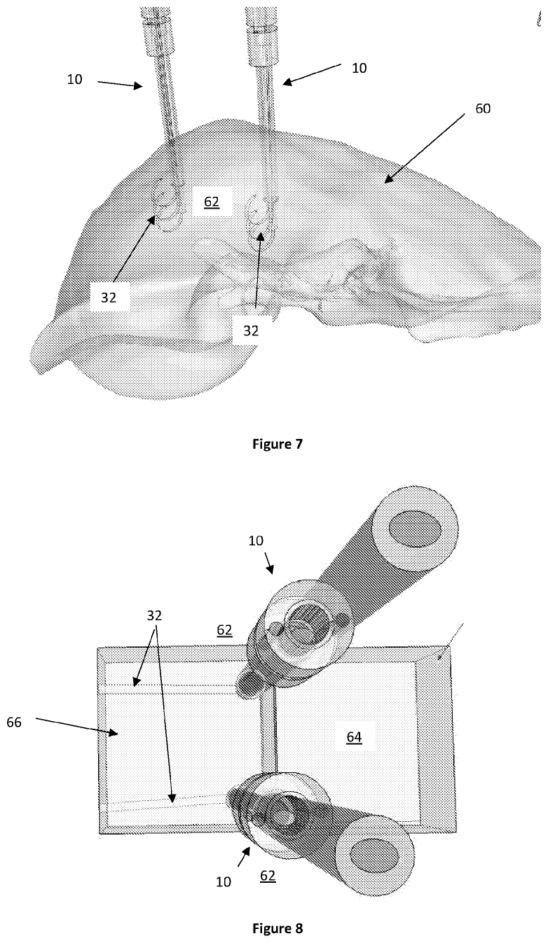

Once the first ablation has been completed, the operator would retract the electrodes 32, 32 back into their respective sleeves 22, 22, release the pin and recess type couplings 70, 70 between the sheath abutting portion 24 and sheath cap 40 and then rotate the probe 20 within the sheath 12 by a desired amount (it may be advisable to retract the probe slightly, so that its distal end 26 retracts into the sleeve 12 before being rotated). In FIG. 7, for example, the electrodes 32, 32 have been partially deployed in a direction opposite to that shown in FIG. 5 (i.e. the probe 20 was rotated through an angle of 180°). In this configuration, the pin and recess type couplings 70, 70 are again able to be used to lock the probe 20 and sheath 12 in this relative orientation. Although not shown, it will be appreciated that providing four pin and recess type couplings similar to those depicted, evenly spread around the device would result in the probe being “lockable” to the sheath at angles of 90°, 180° and 270°. Likewise, other configurations are possible, which might be advantageous for particular ablation devices or multi-step ablation methods.

Referring now to FIG. 8, a second ablation zone 66 is shown between the redeployed electrodes 32, 32. Upon application of an appropriate amount of energy and for an appropriate amount of time, tissue in the second ablation zone 66 is heated from the outside-in to a temperature at which the tissue is completely ablated. As can be seen from FIG. 8, some ablation of tissue surrounding first ablation zone 66 may also occur, but to a lesser extent. Combined ablation zones 64, 66 would be essentially the same at that achievable by one of the multi-electrode array ablation devices disclosed in U.S. Pat. No. 9,060,782 for example, but using an ablation device having a smaller diameter sheath and one more compatible with percutaneous procedures.

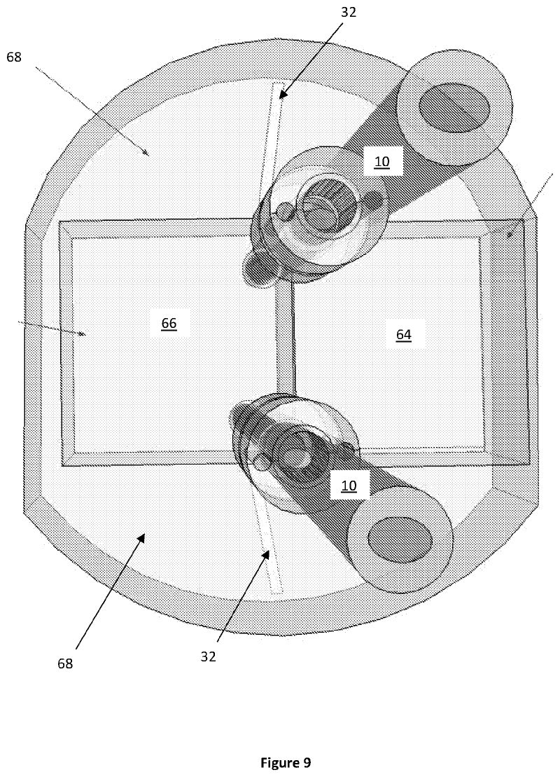

As depicted in FIG. 8, some of tumour 62 may not have been ablated during the first and second ablations (e.g. if tumour 62 is larger in volume than the combined first 64 and second 66 ablation zones or has an irregular shape). In such embodiments, a third ablation may be conducted, as will now be described with reference to FIG. 9. In FIG. 9, the electrodes 32, 32 of the devices 10, 10 of FIGS. 6 and 8 have been redeployed at angles of about 90° and 270°, respectively, from the original ablation (i.e. 0°). Upon application of an appropriate amount of energy to the electrodes 32, 32, tissue in the third ablation zone 68 is heated because the electrical current is not able to pass directly between the electrodes 32, 32 due to the necrotic tissue in the first and second ablation zones 64, 66 being non-conductive. Instead, the current has to pass around the first and second ablation zones 64, 66, thereby creating an oval-shaped third ablation zone 68 and resulting in a combined ablation zone 64, 66, 68 that is larger than tumour 62. In this manner, the device 10 has been operable in a number of steps to ablate a central zone between the sheathes, but also to ablate around an edge of that zone, all without having to reposition the sheathes 12, 12. In effect, smaller devices 10, 10 have been used to ablate a much larger tumour than would previously have been possible using devices double the size.

Although not shown in the Figures, it should be noted that the operator is able to remove one or both of probes 20, 20 from the in situ sheathes 12, 12 and replace that probe with another probe having different characteristics. For example, probes housing larger or smaller electrodes, housing more or less electrodes, housing electrodes formed form different materials or housing electrodes with different electrode configurations can be switched at the operator's discretion and based on observations made during the procedure itself (which is often when a tumour's characteristics first become truly apparent).

The inventors have manufactured prototype tissue ablation devices in accordance with the present invention and as described above as ablation device 10. The results of these laboratory trials are described below.

Tables 1 and 2, set out below, show the results of a first series of experiments using ablation devices having a sheath diameter of 1.6 mm. The first pair of devices have three electrodes which, in their deployed configuration, each have a col diameter of about 1.5 cm. In the results shown below in Table 1, the coil electrodes had a separation of 3 cm. The second pair of ablation devices have three electrodes which, in their deployed configuration, each have a col diameter of about 2 cm. In the results shown below in Table 2, the coil electrodes had a separation of 4 cm.

A first ablation (the “A” ablation, as depicted in FIG. 6 for example), a first and second ablation (“A+B” ablation, as depicted in FIG. 8 for example), where the electrodes were deployed at 0° and then 180°, and a third ablation (“A+B+C” ablation, with the 2 cm electrodes only, as depicted in FIG. 9 for example), where the electrodes were deployed at 0° and then 180° and then finally 90°/270°, were carried out in a fresh calf liver. The liver was then dissected in order for the dimensions of the ablated tissue to be measured.

It will be seen that the “B” ablation added approximately 1 cm to all three dimensions when using the 1.5 cm coil electrodes. The 2 cm coil electrodes “C” ablation added approximately 2 cm in all dimensions additional to the “A+B” sequence.

TABLE 1
Ablations using the device with three electrodes having 1.5 cm coils
3 cm probe separation
Grey zone ablation only
70 watts

| | Vertical | Horizontal | Length |
|---|---|---|---|
| Experiment | (cm) | (cm) | (cm) |
| “A” Ablation | 3 | 3 | 4 |
| “A” Ablation (repeat) | 4 | 3.5 | 4 |
| “A + B” Ablation | 5 | 5 | 5 |
| “A + B” Ablation (repeat) | 5 | 5 | 4.5 |

TABLE 2
Ablations using the device with three electrodes having 2 cm coils
4 cm probe separation
Grey zone ablation only
80 watts

| | Vertical | Horizontal | Length |
|---|---|---|---|
| Experiment | (cm) | (cm) | (cm) |
| “A” Ablation | 5 | 4 | 4 |
| “A + B” Ablation | 5 | 5 | 5.5 |
| “A + B” Ablation (repeat) | 5 | 5 | 4 |
| “A + B + C” Ablation | 7 | 6 | 7 |
| “A + B + C” Ablation (repeat) | 7.5 | 6 | 7 |

Tables 3 to 6, set out below, show the results of a second series of experiments using two pairs of ablation devices in accordance with an embodiment of the present invention. The first devices had a 1.6 mm diameter shaft and 3×1.5 cm electrode coils (referred to below as the “3×1.5” device) deployed from one side of the devices' probes. The second devices had a 1.6 mm diameter shaft and 4×2 cm electrode coils (referred to below as the “4×2” device) deployed from one side of the devices' probes. Multiple deployment angles were used to test which rotation sequence would yield the most constant spherical shape and size.

The performance of the ablation devices of the present invention was compared with that of the InCircle™ Monarch (RFA medical, Inc., Fermont, Calif., USA). The electrodes of the InCircle devices deploy into electrode arrays that are shaped like a rectangle in cross-section and have dimensions of 4×4 cm or 3×3 cm, depending on the model. In the results discussed below, these conventional devices are referred to as the “4×4” and “3×3” devices, respectively. The shaft diameter for both devices is 2.7 mm, which is more than 50% larger than the shafts of the 3×1.5 and 4×2 devices. When the InCircle device's electrodes are deployed, two opposite sets of circular electrode antennas are deployed within the parenchyma of the liver. The rationale behind this deployment method is to increase the surface area of electrodes in the intended ablation field, and this is known to increase the zone and quality of ablation.

Ablations were carried out on bovine liver using the technique described below. A total of 37 ablations in bovine liver and 4 in perfused liver were performed. The bovine livers were obtained fresh on the day of the experiments and were immersed into warm water at 37-40 degrees. The core temperature of the liver was measured with a thermocouple until 37° C. was reached. After that the liver specimen was placed into a container and experiments commenced and recorded.

Perfused bovine liver experiments were also conducted. Livers were obtained fresh from an abattoir, immediately flushed with heparinized kreb's solution with a concentration of 3000 iu of heparin/L and kept on ice immersed in kreb's solution. The livers were perfused using kreb's solution as a perfusate at a rate of 0.8 ml/gram/minute, with a Maquet centrifugal pump being used for perfusion. The perfusate was circulated in a hot water bath at 37° C. and ablations started after the liver temperature reached 36° C.±1. Ultrasound guidance was used in order to avoid insertion into major vessels.

All ablations were performed using the generator's power control mode which delivers the RFA current until complete tissue impedance is achieved. Power was set to the wattage noted in the Tables set out below, and the electrodes were tested at the spacing distances noted in the Tables (with the intended distance being marked at the liver tissue and a spacer used to maintain the intended distance after electrodes were inserted).

The InCircle devices (i.e. the “4×4” and “3×3” devices) were deployed and tested on the liver specimens to provide a benchmark for comparison. The 3×1.5 and 4×2 ablation devices of the present invention was then tested on the same liver. Times of every ablation position was recorded after full impedance was reached and, after all intended ablation positions were performed, the ablated liver was examined, dissected, measured and photographed.

The liver temperature was taken at the start of every experiment using thermocouples. Time for each ablation was registered by the generator, total ablation time was calculated by calculating the sum of time for the steps involved, depending on the positions intended. The ablated liver specimen was first bisected along the line of sight, longitudinal (x axis) and horizontal (y axis) dimensions were measured with a linear centimetre ruler and photographed. Then the specimen was transected perpendicular to the line of sight and the depth (z axis) was measured

The electrode deployment configurations of the devices of the present invention were:
A. Electrodes are initially deployed at 070°
B. Electrodes are retracted, probes rotated in situ, and then the electrodes are deployed again at 180°/180°
C. Electrodes are retracted, probes rotated in situ, and then the electrodes are deployed again at 90°/270°
D1. Electrodes are retracted, probes rotated in situ, and then the electrodes are deployed again at 135°/225°
D2. Electrodes are retracted, probes rotated in situ, and then the electrodes are deployed again at 45°/315°

A total of 37 ablations in bovine liver were performed. The InCircle Monarch (3×3 cm model) was used at 4.5 cm spacing on 70 watts and the InCircle Monarch (4×4 cm model) was used at 4.5 cm spacing on 80 watts to benchmark the results. A series of combinations of rotating sequential ablations were performed using the 3×1.5 cm and 4×2 cm devices of the present invention, at different power settings and spacing distances.

Table 3 shows the results for the ablations performed using the devices of the present invention, along with the benchmark ablations that were performed with the InCircle Monarch. The A+B+D1+D2 rotating sequential ablation resulted in the biggest spherical ablations, which sizes were 5.1×5.1×6.8 for the 3×1.5 cm model compared to 4.5×4.5×4.75 for the 3×3 cm model in 2.3 minutes less than the 3×3 model. The same sequence for the 4×2 cm model resulted in 6×6.25×7 cm compared to 6×5×6.25 cm for the 4×4 cm model and 3.95 minutes faster than the original model.

These results demonstrate that the ablation of tumours up to 5 cm using devices in accordance with the present invention is safe and feasible. The difference between the ablation devices of the present invention and the corresponding InCircle Monarch are shown in Table 4.

TABLE 3
Ablation results in bovine liver

| | | | | | Mean | Mean size cm | | | Mean volume |
|---|---|---|---|---|---|---|---|---|---|
| electrode | spacing | Ablation type | n | power | time | x | y | z | cm³ |
| 3 × 3 | 4.5 | Single ablation | 2 | 70 | 15.7 | 4.5 | 4.5 | 4.75 | 402.91 |
| 3 × 1.5 | 3 | A | 2 | 70 | 4.45 | 3.5 | 3.25 | 4 | 190.59 |
| 3 × 1.5 | 3 | A + B | 4 | 70 | 7.2 | 4.5 | 4.15 | 4.37 | 341.85 |
| 3 × 1.5 | 3 | A + B | 1 | 90 | 5.9 | 4 | 4 | 4 | 268.08 |
| 3 × 1.5 | 3 | A + B | 1 | 110 | 2.8 | 3 | 3 | 3.5 | 131.95 |
| 3 × 1.5 | 3 | A + B + C | 3 | 70 | 9.2 | 5.5 | 4.1 | 5.1 | 481.73 |
| 3 × 1.5 | 3 | A + B + D1 + D2 | 3 | 70 | 12.4 | 4.8 | 4.1 | 5 | 412.18 |
| 3 × 1.5 | 3 | A + B + C + D1 + D2 | 2 | 70 | 11.25 | 4 | 3.5 | 4.5 | 263.89 |
| 3 × 1.5 | 4 | A + B + C | 2 | 70 | 13.9 | 4.75 | 5 | 6.5 | 646.64 |
| 3 × 1.5 | 4 | A + B + D1 + D2 | 3 | 70 | 13.4 | 5.1 | 5.1 | 6.8 | 740.86 |
| 3 × 1.5 | 4 | A + B + C + D1 + D2 | 1 | 70 | 19.8 | 5 | 5 | 7 | 733.04 |
| 4 × 4 | 4.5 | Single ablation | 2 | 80 | 25.8 | 6 | 5 | 6.25 | 785.4 |
| 4 × 2 | 4 | A | 2 | 80 | 7 | 5 | 4 | 4 | 335.1 |
| 4 × 2 | 4 | A + B | 2 | 80 | 9.15 | 5 | 5 | 4.75 | 497.42 |
| 4 × 2 | 4 | A + B + C | 2 | 80 | 19.3 | 7.25 | 6 | 7 | 1275.49 |
| 4 × 2 | 4.5 | A + B | 2 | 80 | 13.8 | 5.25 | 5.25 | 5.2 | 600.36 |
| 4 × 2 | 4.5 | A + B + D1 + D2 | 2 | 80 | 21.85 | 6 | 6.25 | 7 | 1099.56 |
| 4 × 2 | 4.5 | A + B + D1 + D2 | 1 | 70 | 33.8 | 7 | 7 | 7.5 | 1539.38 |

TABLE 4
Comparison of ablation results in bovine liver

| | | | | | | | | Mean volume |
|---|---|---|---|---|---|---|---|---|
| | | | | Mean | Mean size difference cm | | | difference |
| electrode | spacing | Ablation type | power | time | x | y | z | cm³ |
| 3 × 3 | 4.5 | Single ablation | 70 | 15.7 | 4.5 | 4.5 | 4.75 | 402.91 |
| 3 × 1.5 | 3 | A | 70 | −11.25 | −1 | −1.25 | −0.75 | −212.32 |
| 3 × 1.5 | 3 | A + B | 70 | −8.5 | 0 | −0.35 | −0.38 | −61.06 |
| 3 × 1.5 | 3 | A + B | 90 | −9.8 | −0.5 | −0.5 | −0.75 | −134.83 |
| 3 × 1.5 | 3 | A + B | 110 | −12.9 | −1.5 | −1.5 | −1.25 | −270.96 |
| 3 × 1.5 | 3 | A + B + C | 70 | −6.5 | +1 | −0.4 | +0.35 | +78.83 |
| 3 × 1.5 | 3 | A + B + D1 + D2 | 70 | −3.2 | +0.3 | −0.4 | +0.25 | +9.27 |
| 3 × 1.5 | 3 | A + B + C + D1 + D2 | 70 | −4.45 | −0.5 | −1 | −1.25 | −139.02 |
| 3 × 1.5 | 4 | A + B + C | 70 | −1.8 | +0.25 | +0.5 | +1.75 | +243.73 |
| 3 × 1.5 | 4 | A + B + D1 + D2 | 70 | −2.3 | +0.6 | +0.6 | +2.05 | +337.95 |
| 3 × 1.5 | 4 | A + B + C + D1 + D2 | 70 | +4.1 | +0.5 | +0.5 | +2.25 | +330.14 |
| 4 × 4 | 4.5 | Single ablation | 80 | 25.8 | 6 | 5 | 6.25 | 785.4 |
| 4 × 2 | 4 | A | 80 | −18.8 | −1 | −1 | −2.25 | −450.3 |
| 4 × 2 | 4 | A + B | 80 | −16.65 | −1 | 0 | −1.5 | −287.98 |
| 4 × 2 | 4 | A + B + C | 80 | −6.5 | +1.25 | +1 | +0.75 | +490.09 |
| 4 × 2 | 4.5 | A + B | 80 | −12 | −0.75 | +0.25 | −1.05 | −185.04 |
| 4 × 2 | 4.5 | A + B + D1 + D2 | 80 | −3.95 | 0 | +1.25 | +0.75 | +313.6 |
| 4 × 2 | 4.5 | A + B + D1 + D2 | 70 | +8 | +1 | +2 | +1.25 | +753.98 |

A total number of four experiments were performed in perfused liver. Table 5 shows the results of perfused liver experiments by the 3×1.5 cm device (of the present invention). The results verify the outcome of bench experiments, as the ablation sizes achieved were not decreased by perfusion. They did need more time to achieve full impedance, but the inventors believe that this resulted in better, larger and more spherical ablation zones. The improvement from the original InCircle Monarch 3×3 cm is shown in Tables 5 and 6.

TABLE 5
Ablation results in perfused bovine liver

| | | | | Mean | Mean size | | | |
|---|---|---|---|---|---|---|---|---|
| Electrode | Spacing | Ablation type | power | time | x | y | z | Mean volume |
| 3 × 3 | 3 | Single ablation | 70 | 9.2 | 5.5 | 4.5 | 5 | 518.36 |
| 3 × 1.5 | 3 | A | 70 | 4.2 | 3.5 | 3.5 | 4 | 205.25 |
| 3 × 1.5 | 3 | A + B | 70 | 10.5 | 5 | 5 | 5.5 | 575.96 |
| 3 × 1.5 | 3 | A + B + D1 + D2 | 70 | 27 | 7 | 7 | 5.5 | 1128.88 |

TABLE 6
Comparison of ablation results in perfused bovine liver

| | | | | Mean | Mean size difference cm | | | Mean volume |
|---|---|---|---|---|---|---|---|---|
| Electrode | Spacing | Ablation type | power | time | x | y | z | difference cm³ |
| 3 × 3 | 3 | Single ablation | 70 | 9.2 | 5.5 | 4.5 | 5 | 518.36 |
| 3 × 1.5 | 3 | A | 70 | −5 | −2 | −1 | −1 | −313.11 |
| 3 × 1.5 | 3 | A + B | 70 | +1 | −0.5 | +0.5 | +0.5 | +57.6 |
| 3 × 1.5 | 3 | A + B + D1 + D2 | 70 | +17.8 | +2 | +2.5 | +0.5 | +610.52 |

All ablated liver tissue was examined, bisected then transected. All ablation zones were homogenous with no fissures or inadequately ablated areas or spots.

Based on the experiments described herein, the method of sequential rotating ablations appear to result in larger ablations while requiring less time than for the InCircle Monarch. Whilst overlapping ablation zones may be thought of as an inefficient use of RF energy, the inventors note the significant advantages of the shafts of the devices of the present invention not needing to be removed, with the electrodes simply being withdrawn within from the treatment zone, rotated and redeployed. Furthermore, no areas of untreated liver tissue were seen in the inventors' experiments, in contrast to overlapping monopolar ablations.

In summary, the inventors' experiments have identified a novel technique that can decrease the size of the ablation device's shafts to 1.6 mm, whilst still achieving up to 7 cm ablations, using the ablation protocols described herein. This device is ideal for interventional radiology physicians to allow large no touch ablations with small electrodes for use in open or laparoscopic surgeries or percutaneous interventions.

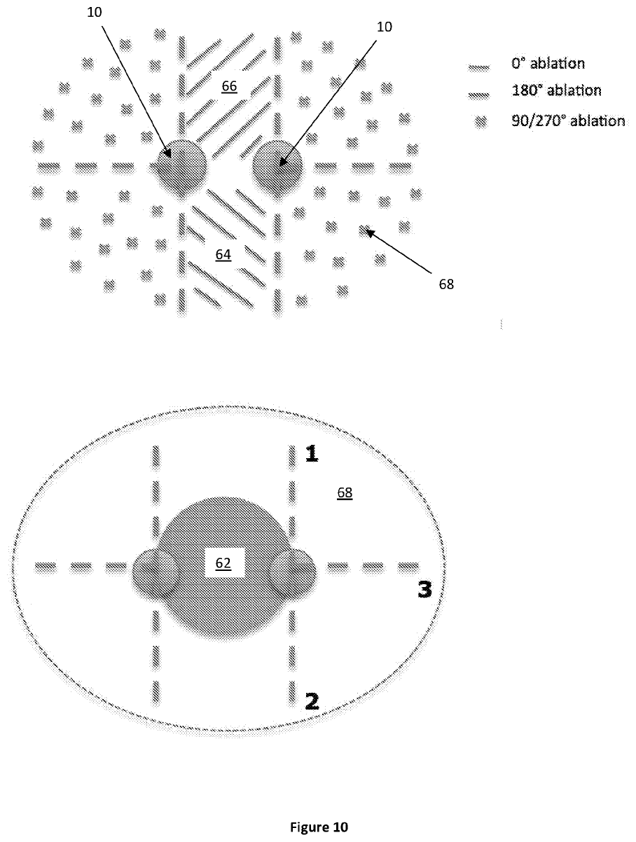

Referring now to FIG. 10, an alternative depiction of the combined ablations achieved by performing ablations with electrodes deployed at angles of 0°, 180° and 90° (i.e. as per FIG. 9) is shown. As can be seen, the resultant ablation is generally egg-shaped, with the lateral)(90°/270° deployment of the electrodes 32, 32 creating a lateral extension of the ablation.

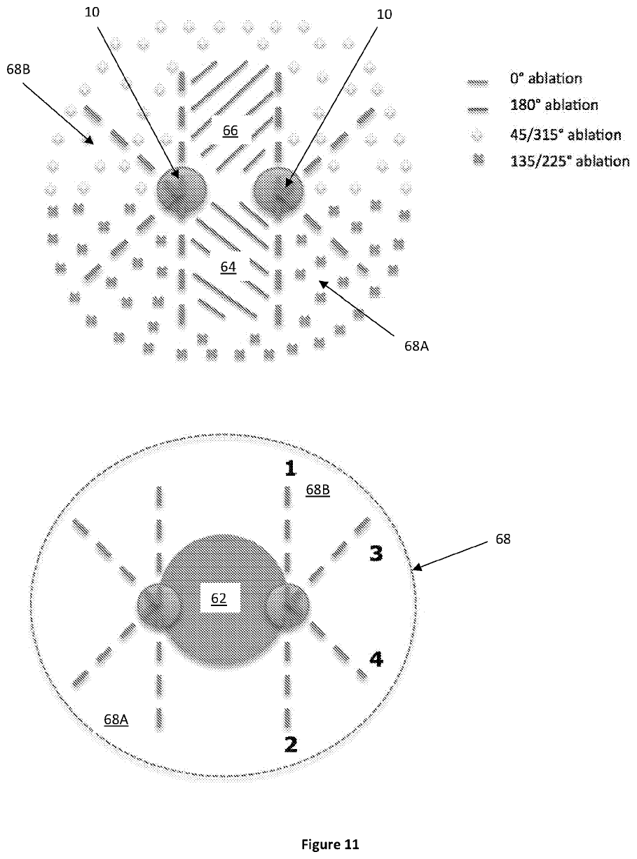

FIG. 11 shows depicts the combined ablations achieved by performing four ablations with the electrodes deployed at angles of 0°, 180°, 45/315° and 135/225°, which results in a more spherical ablation. As can be seen in the Figures, the 45/315° electrode deployment angles ablates tissue 68B around and above (as shown in the Figure) the central ablation zone (defined by ablations 64, 66), and the 135/225° electrode deployment angles ablates tissue 68A around and below (as shown in the Figure) the central ablation zone 64, 66.

Choosing between the “edge boosts” around the central ablation zone (64, 66) depicted in FIGS. 10 and 11 depends on factors such as the location of the tumour 62. If the tumour 62 is close to the edge of liver 60 or a vessel in the liver, doing a 90°/270° ablation (i.e. that depicted in FIG. 10) might deploy one of the electrodes 32 outside of the liver 60 or into the vessel, etc. In such situations, choosing the “closer” 45°/135° ablation (i.e. that depicted in FIG. 11) would be more appropriate.

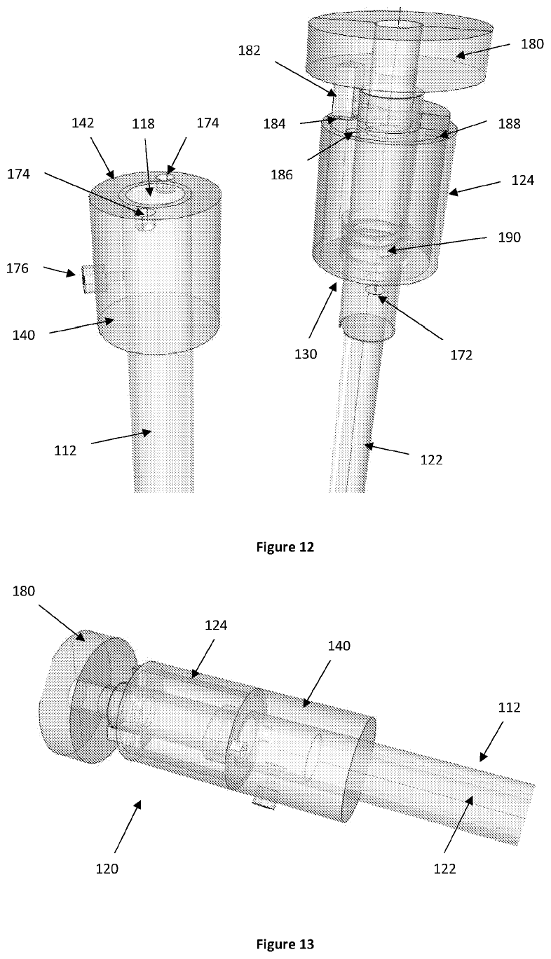

Referring now to FIGS. 12 and 13, the coupling between a probe 120 and sheath 112 of a device 110 (shown assembled in FIG. 13) in accordance with another embodiment of the invention is shown in greater detail. Probe 120 and sheath 112 are similar to the probe 20 and sheath 12 described above, with the main points of difference being noted below. Sheath 112 is shown on the left in FIG. 12 and includes a removable sheath cap 140 that is fastenable to the sheath 112 via locking pin 176. The outwardly facing surface 142 of the sheath cap 140 is clearly visible, as are recesses 174, 174 on opposing sides of the surface 142. Recesses 174, 174 are configured to receive corresponding pins 172, 172 on the inwardly facing surface 130 of the probe's sheath abutting portion 124, and together provide a means for ensuring that the probe 120 and sheath 112 are in a correct alignment (i.e. as shown in FIG. 13).

Probe 120 is shown on the right in FIG. 12 and includes a twistable knob in the form of dial 180. Dial 180 is operable to change the orientation of the probe 120 (specifically, its sheath 122 and hence the angle at which the electrodes (not shown) will deploy) with respect to the sheath 112. An alignment member 182 that depends from the dial 180 is alignable with apertures 184, 186 and 188 on an upper surface of the sheath abutting portion 124. Alignment of the member 182 with apertures 184, 186 and 188 corresponds, in this embodiment, to electrode deployments at 0°, 90° and 180°. A spring and circlip 190 may also be provided to hold the probe's sleeve 122 within sheath abutting portion 124 and to provide for positive indexing during rotation of the dial 180 (i.e. the alignment member 182 is urged into a respective aperture 184, 186 or 188 by the spring 190).

FIG. 13 shows the probe 120 and sheath 112 in an assembled configuration. As would be appreciated, the positions of pins 172 and recesses 174 enable probe 120 shown in FIG. 13 to have two orientations with respect to sheath 112, which will result in the electrodes being deployed at an angle of 180° to each other. Fine tuning of the dial 180 may be used to adjust the angle of deployment of the electrodes (not shown) by angles of 90°, in the manner described above.

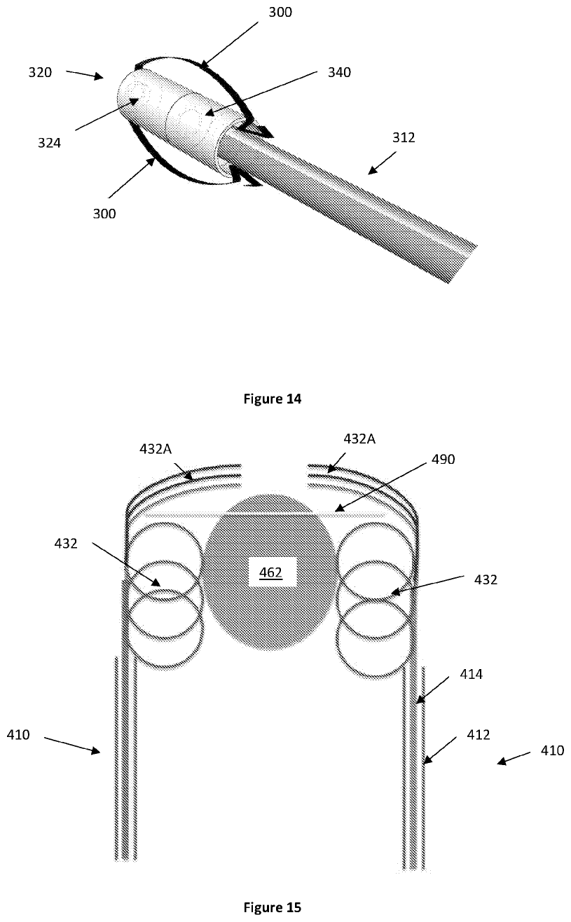

Referring now to FIG. 14, shown is an alternative mechanism via which a probe may be releasably coupled to a sheath in devices in accordance with other embodiments of the present invention. In FIG. 14, for example, clips 300, 300 may be used to clip the probe's sheath abutting portion 324 to the sheath's cap 340 once in the desired orientation. Although not shown, the side walls of the sheath abutting portion 324 and sheath cap 340 may include indicia (e.g. laterally arranged lines spaced around a periphery of the sheath abutting portion and sheath cap) which may be visually aligned in order to define a respective orientation of the probe 320 with respect to the sheath 312 before locking them together using the clips 300, 300.

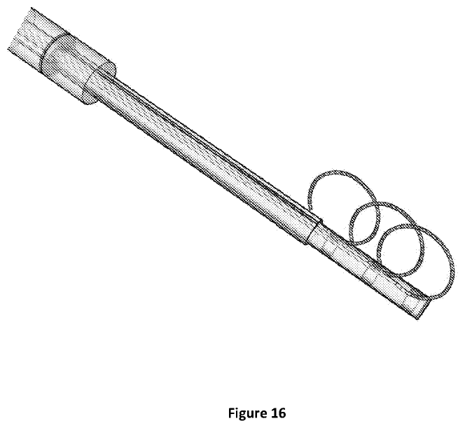

Referring now to FIG. 15, a schematic drawing of a multi-stage ablation process involving the use of different types of probes/electrodes is shown. Probes 410, 410, each of which have a three coil ablation electrode 432 are positioned to either side of a tumour 462. The first ablations carried out with the coiled electrodes 432, 432 will ablate the portion of the tumour 462 below the line 490. However, further investigation by the operator during the procedure may indicate that the tumour 462 extends beyond the combined ablation zone of the coiled electrodes 432, 432, even following a 180 degree and a 90 degree ablation of the type described above.

In such circumstances, each of the coiled electrodes 432 may be retracted into the probe's sleeve 422 and the probe 420 removed from sheath 412. Subsequently, a new probe 420A having electrodes 432A that deploy into a configuration that extends beyond and effectively encases the tumour 462 may be inserted into the sheathes 412, 412 and so-deployed. Ablation using electrodes 432A, 432A would heat and destroy the portion of tumour 462 above the line 490, thereby ablating the tumour in its entirety in a single procedure and using only two percutaneously inserted sheathes 412, 412. The electrodes 432A are shown having 3 similarly shaped and curved electrodes but could, in some embodiments be a single electrode and could have other tumour encompassing configurations.

Finally, FIG. 16 shows an alternative embodiment of an ablation device in accordance with the present invention, in which the electrodes are shown in a fully deployed configuration and in the form of round wire coils. The electrodes are deployed from three apertures spaced in line along the probe's sleeve and together define a substantially planar electrode coil array to the side of the probe.

In summary, the invention relates to devices and methods for ablating biological tissue. It will be appreciated from the foregoing disclosure that the present invention provides a number of new and useful results. For example, specific embodiments of the present invention may provide one or more of the following advantages:
a smaller ablation device than those presently on the market can be used to produce ablations having a volume comparable to, or larger than, those producible by the larger devices presently on the market;
the small gauge of the sheath enables use of the device in percutaneous procedures, lessening the complexity of the procedure and reducing possible complications;
choice of electrode size, shape and configuration, as well as its angle of deployment provides the operator with an unprecedented level of control over the ablation volume, even after the procedure has commenced; and
slight misplacements of the sheath at the start of a procedure can be remedied, without having to restart the procedure, by a corresponding adjustment to the angle of deployment and/or deployed electrode size or configuration.

It will be understood to persons skilled in the art of the invention that many modifications may be made without departing from the spirit and scope of the invention. All such modifications are intended to fall within the scope of the following claims.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A tissue ablation device comprising:
a sheath for positioning within body tissue, the sheath comprising a distal end, a proximal end and a lumen extending therebetween; and
a probe comprising an elongate portion configured to be slidably received in the lumen, the elongate portion housing an electrode that is deployable from a distal end of the elongate portion into a substantially planar deployed configuration when the distal end of the elongate portion is located at or beyond the distal end of the sheath,
whereby an angle of deployment of the electrode from the distal end of the probe is selectable by orientating the probe with respect to the sheath.

2. The tissue ablation device of claim 1, wherein the probe further comprises a sheath abutting portion configured for receipt at the proximal end of the sheath when the distal end of the elongate portion is located at or beyond the distal end of the sheath.

3. The tissue ablation device of claim 2, wherein the sheath abutting portion and the proximal end of the sheath comprise means for indicating a relative orientation therebetween.

4. The tissue ablation device of claim 2, wherein the sheath abutting portion and the proximal end of the sheath comprise visual or tactile means for indicating a relative orientation therebetween.

5. The tissue ablation device of claim 2, wherein the sheath abutting portion and the proximal end of the sheath comprise surfaces that abut one another in use, the respective surfaces comprising indicia to visually show the relative orientation therebetween.

6. The tissue ablation device of claim 2, wherein the sheath abutting portion and the proximal end of the sheath comprise surfaces that abut one another in use, the respective surfaces comprising complimentary protrusions and recesses configured to mate when the sheath abutting portion and the proximal end of the sheath are orientated at predefined angles.

7. The tissue ablation device of claim 6, wherein the predefined angles are about 0°, 90°, 180° and 270°.

8-10. (canceled)

11. The tissue ablation device of claim 1, wherein the electrode comprises a plurality of electrodes, each electrode assuming a different deployed configuration upon deployment.

12. The tissue ablation device of claim 11, wherein the plurality of electrodes are each independently deployable through a respective orifice at the end of and/or along a side of the elongate portion at the distal end of the probe.

13. The tissue ablation device of claim 1, wherein the probe for use in the device is selectable from a plurality of available probes, the electrodes in the available probes being configured to assume selectable deployed configurations.

14. The tissue ablation device of claim 1, further comprising a deployment actuator which is operable to deploy the electrode from the distal end of the probe.

15. The tissue ablation device of claim 14, wherein the deployment actuator is operable to advance and retract the electrode between the deployed configuration and a retracted configuration.

16. (canceled)

17. A method for ablating tissue within an ablation zone in a patient's body, the method comprising:
(a) positioning the sheathes of two tissue ablation devices of claim 1 in the patient, at least a portion of the ablation zone being located between the sheathes;
(b) orientating the probes of the tissue ablation devices with respect to the sheathes whereby the electrodes will deploy in a first configuration;
(c) deploying the electrodes in the first configuration and ablating tissue between the so-deployed electrodes to form a first ablated portion;
(d) retracting the electrodes back into the respective probes;
(e) reorientating the probes with respect to the sheathes whereby the electrodes will deploy in a second configuration;
(f) deploying the electrodes in the second configuration and ablating tissue between the so-deployed electrodes to form a second ablated portion;
(g) repeating steps (d) to (f) until the combined ablated portions define the ablation zone; and
(h) withdrawing the sheathes from the patient.

18. A method for ablating tissue within an ablation zone in a patient's body, the method comprising:
(a) positioning the sheath of a tissue ablation device of claim 1 in the patient at the ablation zone;
(b) orientating the probe of the tissue ablation device with respect to the sheath whereby the electrode will deploy in a first configuration;
(c) deploying the electrode in the first configuration and ablating tissue to form a first ablated portion;
(d) retracting the electrode back into the probe;
(e) reorientating the probe with respect to the sheath whereby the electrode will deploy in a second configuration;
(f) deploying the electrode in the second configuration and ablating tissue to form a second ablated portion;
(g) repeating steps (d) to (f) until the combined ablated portions define the ablation zone; and
(h) withdrawing the sheath from the patient.

19. The method of claim 18, wherein ablation occurs between the deployed electrode and a ground plate, between deployed electrodes of the device which have an opposite polarity or between the deployed electrode and a portion of the device having an opposite polarity.

20. The method of claim 17, wherein the angle between the first and second configurations is 180°.

21. The method of claim 17 comprising three ablations, wherein the angle between the first and second configurations is 180° and the angle between the second and third configurations is 90°.

22. The method of claim 17 comprising the additional step of replacing the probe or one of the probes with a probe having a different electrode between ablations.

23. The method of claim 22, wherein the different electrode differs in one or more of the size and shape of its deployed configuration.

24. The method of claim 17 comprising percutaneously positioning the or each sheath in the patient.

25-28. (canceled)
